(19) United States
(12) Patent Application Publication
(10) Pub. No.: US 2023/0043432 A1
(43) Pub. Date: Feb. 9, 2023

(54) INPUT DEVICES FOR ROBOTIC SURGICAL SYSTEMS

(71) Applicant: Covidien LP, Mansfield, MA (US)

(72) Inventor: Jaimeen V. Kapadia, Cambridge, MA (US)

(21) Appl. No.: 17/878,137

(22) Filed: Aug. 1, 2022

Related U.S. Application Data
(60) Provisional application No. 63/229,203, filed on Aug. 4, 2021.

(51) Int. Cl. A61B 34/00; A61B 34/37; B25J 13/06

(52) U.S. Cl. CPC A61B 34/74; A61B 34/37; B25J 13/06

(57) ABSTRACT

A surgical robot system includes a robot system including a robotic arm having a surgical instrument coupled thereto, a surgical console including an input device, and a control unit configured to control the robotic arm based on input commands from the input device. The input device includes a gimbal including first, second, and third links, a handle controller coupled to the first link of the gimbal, and a gimbal-mounted actuator assembly including a first actuator supported on the gimbal. The first actuator is configured to effect a function of the surgical instrument coupled to the robotic arm.

### CROSS-REFERENCE TO RELATED APPLICATIONS

This application claims priority to, and the benefit of, U.S. Provisional Patent Appl. No. 63/229,203, filed Aug. 4, 2021, the entire contents of which are hereby incorporated by reference herein.

### FIELD

This disclosure generally relates to robotic surgical systems. In particular, this disclosure relates to an input device of a robotic surgical system that enables a clinician to control a robotic arm of the robotic surgical system during a surgical procedure.

### BACKGROUND

Robotic surgical systems are currently being used in minimally invasive medical procedures. Some robotic surgical systems include a surgical console controlling one or more robotic arms that each includes a surgical instrument having an end effector coupled to and actuated by the respective robotic arm. The surgical console allows a surgeon to manipulate the robotic arm and the surgical instrument that acts on a patient.

### SUMMARY

The robotic surgical system of this disclosure includes one or more input devices that are used by a clinician to control one or more robotic arms of the robotic surgical system. The input devices include one or more actuators for effecting one or more functions of the robotic arms.

In aspects of the disclosure, a robotic surgical system includes a robot system including a robotic arm having a surgical instrument coupled thereto, a surgical console including an input device, and a control unit configured to control the robotic arm based on input commands from the input device. The input device includes a gimbal including first, second, and third links, a handle controller coupled to the first link of the gimbal, and a gimbal-mounted actuator assembly including a first actuator supported on the gimbal. The first actuator is configured to effect a function of the surgical instrument coupled to the robotic arm.

The first actuator of the gimbal-mounted actuator assembly may be supported on the first link of the gimbal. In some aspects, the first link of the gimbal includes a first segment extending distally from the handle controller and a second segment extending downwardly from the first segment, and the first actuator of the gimbal-mounted actuator assembly is disposed on the first segment. In certain aspects, the gimbal-mounted actuator assembly includes a second actuator supported on the second segment of the first link.

In some aspects, the first link of the gimbal includes a third segment extending proximally from the second segment and under the handle controller. In certain aspects, the gimbal-mounted actuator assembly includes a third actuator supported on the third segment. In some aspects, the first link of the gimbal includes a fourth segment extending downwardly from the third segment, and the gimbal-mounted actuator assembly includes a fourth actuator supported on the fourth segment.

The handle controller may include a shaft and pinchers disposed on opposed sides of the shaft. In some aspects, the input device of the surgical console further includes a handle-mounted actuator assembly including a first actuator supported on the shaft of the handle controller.

The first actuator of the gimbal-mounted actuator assembly may be selected from the group consisting of push buttons, touch pads, rotation knobs, toggle switches, slide switches, rocker switches, rotary switches, trackpads, trackballs, and joysticks.

In aspects of the disclosure, a surgical console for a surgical robot system includes a control arm assembly including a plurality of input devices. At least one of the plurality of input devices includes a gimbal including first, second, and third links, a handle controller coupled to the first link of the gimbal, and a gimbal-mounted actuator assembly including a first actuator supported on the gimbal.

The first actuator of the gimbal-mounted actuator assembly may be supported on the first link of the gimbal. In some aspects, the first link of the gimbal includes a first segment extending distally from the handle controller and a second segment extending downwardly from the first segment, and the first actuator of the gimbal-mounted actuator assembly is disposed on the first segment.

The handle controller of the input device may include a shaft and pinchers disposed on opposed sides of the shaft. In some aspects, the input device further includes a handle-mounted actuator assembly including a first actuator supported on the shaft of the handle controller.

In aspects of the disclosure, an input device for a surgical robot system includes a gimbal including first, second, and third links, a handle controller coupled to the first link of the gimbal, and a gimbal-mounted actuator assembly including a first actuator supported on the gimbal.

The first actuator of the gimbal-mounted actuator assembly may be supported on the first link of the gimbal. In some aspects, the first link of the gimbal includes a first segment extending distally from the handle controller and a second segment extending downwardly from the first segment, and the first actuator of the gimbal-mounted actuator assembly is disposed on the first segment.

The handle controller may include a shaft and pinchers disposed on opposed sides of the shaft. In some aspects, the input device further includes a handle-mounted actuator assembly including a first actuator supported on the shaft of the handle controller.

The details of one or more aspects of the disclosure are set forth in the accompanying drawings and the description below. Other features, objects, and advantages of the aspects described in this disclosure will be apparent from the description and drawings, and from the claims.

### BRIEF DESCRIPTION OF THE DRAWINGS

Various aspects of this disclosure are described hereinbelow with reference to the drawings, which are incorporated in and constitute a part of this specification, wherein:

FIG. 1 is a schematic illustration of a robotic surgical system including a robot system, a surgical console having two input devices, and a control unit in accordance with aspects of the disclosure;

FIGS. 2 and 3 are perspective views of a handle controller and a gimbal of one of the input devices of FIG. 1 in accordance with an aspect of the disclosure;

FIG. 4 is a perspective view of a handle controller and a gimbal of an input device in accordance with another aspect of the disclosure; and

FIG. 5 is a perspective view of a handle controller and a gimbal of an input device in accordance with yet another aspect of the disclosure.

### DETAILED DESCRIPTION

Aspects of this disclosure are now described in detail with reference to the drawings in which like reference numerals designate identical or corresponding elements in each of the several views. It is to be understood that the disclosed aspects are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure.

Throughout this description, the term “proximal” refers to a portion of a device, or component thereof, that is closer to a user, and the term “distal” refers to a portion of the device, or component thereof, that is farther from the user. As used herein, the term “patient” should be understood as referring to a human subject or other animal, and the term “clinician” should be understood as referring to a doctor (e.g., a surgeon), nurse, or other care provider and may include support personnel. Directional reference terms, such as “upwardly,” “downwardly,” “under,” “over,” “side,” and the like, are intended to ease description of the components in this disclosure and are not intended to have any limiting effect on the ultimate orientation of a device or any parts thereof.

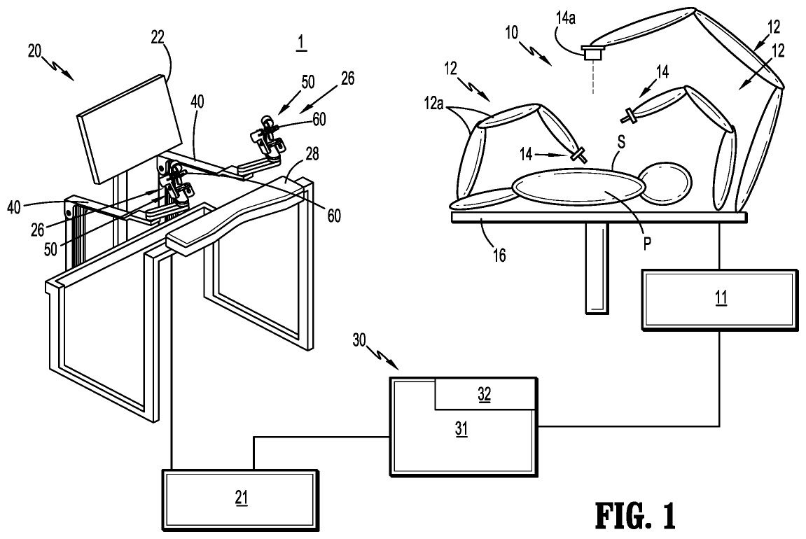

FIG. 1 illustrates an exemplary robotic surgical system 1 in accordance with aspects of the disclosure. The robotic surgical system 1 includes a robot system 10, a surgical console 20, and a control unit or tower 30. Generally, the surgical console 20 receives user input through one or more interface or input devices 26, which are interpreted by the control unit 30 as commands for controlling the robot system 10 (e.g., moving robotic arm(s) 12 and/or effecting function(s) of surgical instrument(s) 14 of the robot system 10).

The robot system 10 generally includes one or more robotic arms 12, with each robotic arm 12 including a surgical instrument 14 removable coupled thereto. In some aspects, one or more of the robotic arms 12 are coupled to a robot base 16 and, in other aspects, one or more of the robotic arms 12 are coupled to a movable cart (not shown). Each of the robotic arms 12 may include linkages 12a that movably supports the surgical instrument 14 which is configured to act on tissue of a patient “P” at a surgical site “S.”

The surgical instruments 14 are configured for use during minimally invasive surgical procedures, however, the surgical instruments 14 may be configured for open surgical procedures. It should be understood that the surgical instruments may be any surgical instrument utilized during a surgical procedure, such as, for example, graspers, scissors, knives, forceps, staplers, cauterizers, aspirators, irrigators, etc. which perform functions for the clinician, for example, grasping a blood vessel, spreading tissue, driving a needle, and/or stapling, dissecting, cauterizing, coagulating, suctioning, irrigating tissue, etc. In some aspects, the surgical instrument 14 is an energy instrument, such as an electrosurgical forceps configured to seal tissue by compressing tissue between jaw members and applying electrosurgical current thereto. In some aspects, the surgical instrument 14 is a surgical stapling instrument, such as a linear or circular surgical stapler configured to grasp and clamp tissue whilst deploying a plurality of tissue fasteners, e.g., staples, and, in certain aspects, cutting the stapled tissue. In some aspects, the surgical instrument 14 is a vision instrument, such as an endoscope configured to provide a video feed for the clinician.

In aspects, as seen in FIG. 1, the robot system 10 includes a plurality of robotic arms 12 with one of the robotic arms 12 including a vision instrument 14a (e.g., a camera) configured to capture images (e.g., video) of the surgical site “S” which are transmitted, in real-time, to a display 22 of the surgical console 20, and the other robotic arms 12 include surgical instruments 14 configured to treat tissue.

The surgical console 20 includes a display 22 which displays a video feed of the surgical site “S” provided by the vision instrument 14a disposed on the robotic arm 12. The display 22 may further display a user interface for controlling the robotic surgical system 1. In some aspects, the surgical console 20 includes first and second displays for separately displaying the video feed and the user interface. The display(s) may be a touchscreen allowing for displaying various graphical user inputs.

The surgical console 20 also includes one or more input devices 26 which are used by a clinician to remotely control the robotic arms 12. The input devices 26 each include a control arm 40, a gimbal or wrist 50, and a handle controller 60. In aspects, the surgical console includes two such input device 26. The handle controllers 60 may be configured for left hand input, right hand input, or universal input (e.g., either left or right handed use). In some aspects, the input devices 26 are of opposite configuration to provide left and right hand inputs. In aspects, the surgical console 20 may include other types of input devices, such as foot pedals (not shown). The surgical console 20 further includes an armrest 28 used to support a clinician's arms while operating the handle controllers 26c.

The handle controllers 60 are operably coupled to the gimbals 50 which, in turn, are operably coupled to the control arms 40. Each input device 26 enables movement of the handle controller 60 with a plurality of degrees of freedom. In aspects, the handle controller 60 is movable about six degrees of freedom, three rotational degrees of freedom and three translational degrees of freedom. In some aspects, the gimbal 50 provides the rotational degrees of freedom and the control arm 40 provides the translational degrees of freedom. It should be understood that at least some of these degrees of freedom may be coupled.

The control unit 30 includes a display 32, which may be a touchscreen, and outputs on graphical user interfaces. The control unit 30 acts as an interface between the robot system 10 and the surgical console 20. In particular, the control unit 30 is configured to control the robotic arms 12, such as to move the robotic arms 12 and the corresponding surgical instruments 14, based on a set of programmable instructions and/or input commands from the surgical console 20, in such a way that the robotic arms 12 and/or the surgical instruments 14 execute desired movement sequences or functions in response to input from the input devices 26.

Each of the robot system 10, the surgical console 20, and the control unit 30 includes a respective computer 11, 21, 31. The computers 11, 21, 31 are interconnected to each other using any suitable communication network based on wired or wireless communication protocols. The computers 11, 21, 31 may include any suitable processor (not shown) operably connected to a memory (not shown). The processor may be any suitable processor (e.g., control circuit) adapted to perform the operations, calculations, and/or set of instructions described in this disclosure.

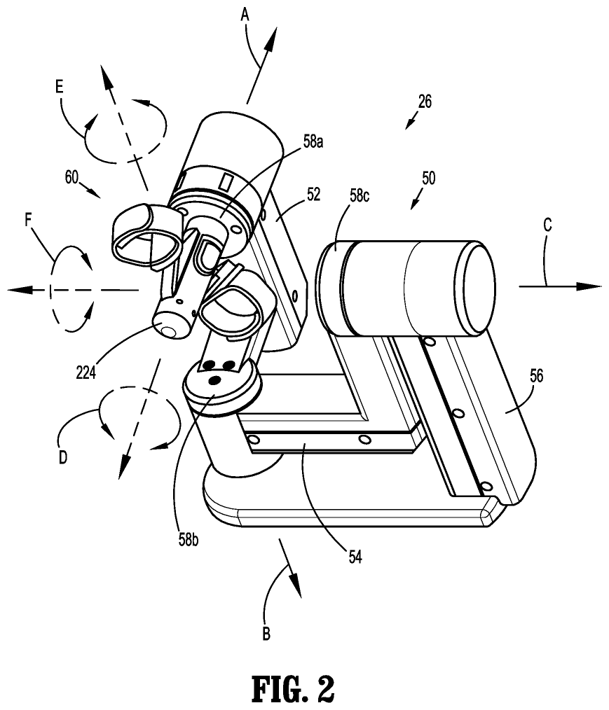

FIG. 2 illustrates the gimbal 50 and the handle controller 60 of one of the input devices 26 of the surgical console 20 (FIG. 1). While two input devices 26 are shown in FIG. 1, the input devices 26 are discussed singularly as they may be identical (e.g., configured for input by either the left or the right hand) or substantially identical (e.g., oppositely configured for left and right handed input). The handle controller 60 is supported by the gimbal 50 and is configured to be grasped by a clinician to control the respective robotic arm 12 (FIG. 1). The gimbal 60 includes first, second, and third links 52, 54, 56 connected together by first, second, and third joints 58a, 58b, 58c (e.g., rotational joints) that enable the handle controller 60 to be translated to a position as suggested by the three straight arrows “A,” “B,” “C,” and is further rotatable to an orientation as suggested by the three curved arrows “D,” “E,” “F”. Specifically, the first link 52 is pivotally coupled to a distal end of the handle controller 60 by the first joint 58a and to the second link 54 by the second joint 58b. The second link 54 is pivotally coupled to the third link 56 by the third joint 58c, and the third link 56 is coupled to the control arm 40 (FIG. 1). In some aspects, the first link 52 includes a connector (not shown) for releasable engagement to the handle controller 60.

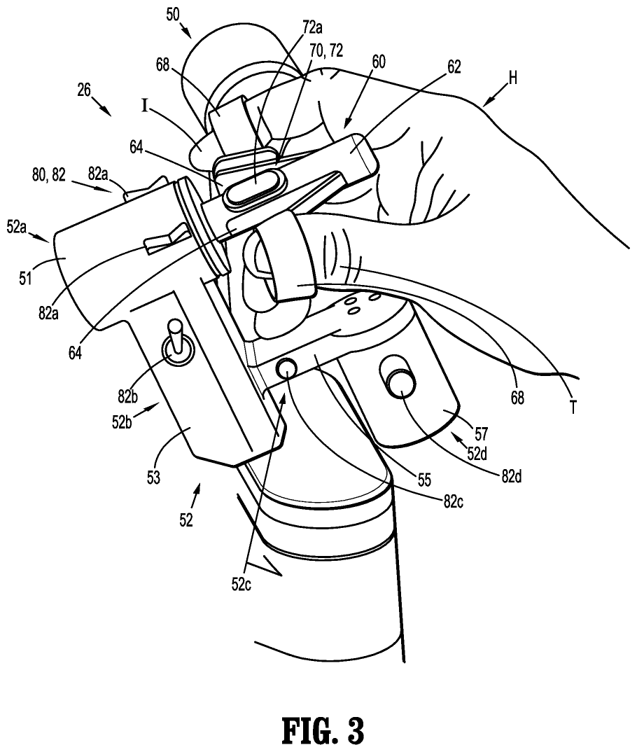

FIG. 3 illustrates a hand “H” of a clinician engaged with the handle controller 60 of the input device 26. The handle controller 60 has a pincher grip configuration in which the clinician grips a handle body or shaft 62 of the handle controller 60 by positioning his or her thumb “T” and index finger “I” over pinchers 64 of the handle controller 60. The thumb “T” and index finger “I” may be held on the pinchers 64 by straps or loops 68. It should be understood that other handle controller styles are envisioned, such as power grip configurations (e.g., a pistol grip), depending upon the preferred or required handle type for a surgical procedure, as is within the purview of those skilled in the art. Accordingly, to move the robotic arm 12 (FIG. 1) and thus, change the orientation of the surgical instrument 14 (FIG. 1) coupled to the robotic arm 12, the clinician moves the handle controller 60 to the desired surgical instrument orientation relative to the image viewed at the surgical console 20 (FIG. 1) as the orientation of the surgical instrument 14 follows the orientation of the handle controller 60.

The handle controller 60 may include a handle-mounted actuator assembly 70 including one or more actuators 72 and/or the gimbal 50 may include a gimbal-mounted actuator assembly 80 including one or more actuators 82 for enabling a clinician to manipulate or control one or more functions of the respective surgical instrument 14 (FIG. 1) supported on the robotic arm 12 (FIG. 1). For example, the actuator(s) 72, 82 may be configured to clamp, grasp, fire, open, close, rotate, thrust, slice, energize, etc. the respective surgical instrument 14 and/or to control the application of energy, force, suction, irrigation, etc. by the surgical instrument 14. In some aspects, the actuator(s) 72, 82 may be configured to control the surgical instrument 14 as well as the visual instrument 14a (FIG. 1) (e.g., to change the field of view), vary a degree of magnification, vary a degree of illumination, change the type of illumination (e.g., white light illumination and/or infra-red illumination) and, in some aspects, the actuator(s) 72, 82 may be configured to toggle between surgical instruments 14 (e.g., when more than two robotic arms 12 are used).

The actuators 72, 82 can have any suitable configuration (e.g., push, touch, rotate, toggle, slide, rock, etc.) for enabling a clinician to manipulate the respective surgical instrument 14 (FIG. 1), as described above. The actuators 72, 82 may be push buttons, touch pads, rotation knobs, toggle switches, slide switches, rocker switches, rotary switches, trackpads, trackballs, joysticks, among other controllers for effecting one or more functions of the surgical instrument 14. The actuators 72, 82 may be single function or multi-function actuators. The actuators 72, 82 may have two discrete positions (e.g., on/off, high/low, fast/slow, etc.), multiple (e.g., three of more) discrete positions (e.g., high/medium/low, fast/normal/slow, etc.), or sliding positions (e.g., to variably control speed, intensity, etc.). The actuator(s) 72 of the handle controller 60 may be supported on any portion of the handle controller 60 and the actuator(s) 82 of the gimbal 50 may be supported on any portion of the gimbal 50.

FIG. 3 illustrates an exemplary configuration of the handle-mounted and gimbal-mounted actuator assemblies 70, 80. The handle controller 60 includes a first actuator 72a supported on the handle body 62 between the pinchers 64. The first actuator 72a is shown as a push button, however, as described above, it should be understood that other types of actuators are envisioned. Further, while the first actuator 72a is shown disposed on the handle body 62 between the pinchers 64, it should be understood that the first actuator 72a may be disposed in other locations (e.g., at a proximal end of the handle body 62). Further still, while the handle-mounted actuator assembly 70 is shown including only the first actuator 72a, it should be understood that the handle-mounted actuator assembly 70 may include more than one actuator 72 (e.g., a plurality of actuators, of the same or different types, disposed along the handle body).

The gimbal 50 includes first, second, third, and fourth actuators 82a, 82b, 82c, 82d supported on the first link 52. The first link 52 includes a first segment 52a extending distally from the handle controller 60 and a second segment 52b extending downwardly from the first segment 52a and thus, is also distal to the handle controller 60. The first link 52 further includes third and fourth segments 52c, 52d disposed under the handle controller 60 in spaced relation relative thereto. The third segment 52c extend proximally from the second segment 52b and the fourth segment 52d extend downwardly from the third segment 52c.

The first segment 52a of the first link 52 supports first actuators 82a disposed in spaced relation relative to each other on an outer surface 51 of the first segment 52a. The first actuators 82a are spaced to correspond with the spacing between the pinchers 64 of the handle controller 60 so that the first actuators 82a can be in-line with the thumb “T” and the index finger “I” of the clinician. In such aspects, the positioning of the first actuators 82a enables the clinician to move (e.g., slide) his or her index finger “I” or thumb “T” distally to actuate either or both of the first actuators 82a. The first actuators 82a are shown as rocker switches, however, as described above, it should be understood that other types of actuators are envisioned for use (e.g., the first actuators 82a may be of the same or different type). Further, while two first actuators 82a are shown disposed in radial spaced relation on the first segment 52a, it should be understood that the number and position of the first actuators 82a may vary (e.g., the first segment 52a may include one first actuator 82a or more than two first actuators 82a and/or the first actuators 82a may be axially and/or radially oriented with respect to each other on the first segment 52a).

The second segment 52b of the first link 52 supports one or more second actuators 82b, the third segment 52c supports one or more third actuators 82c, and the fourth segment 52d supports one or more fourth actuators 82d. Each of the second, third, and fourth actuators 82b, 82c, 82d extend from respective side surfaces 53, 55, 57 of the second, third, and fourth segments 52b, 52c, 52d. In such aspects, the position of the second, third, and fourth actuators 82b, 82c, 82d on the side surfaces 53, 55, 57 of the first link 52 minimizes or prevents inadvertent actuation during use of the handle controller 60. The second actuators 82b are shown as toggle switches, the third actuator 82c is shown as a push button, and the fourth actuator 82d is shown as a rotation knob. It should be understood that any type, number, and position of the second, third, and fourth actuators 82b, 82c, 82d are envisioned depending on the desired functionality of the input device 26. In some aspects, a plurality of each of the second, third, and fourth actuators 82b, 82c, 82d are disposed on opposed side surfaces 53, 55, 57 of the second, third, and fourth segments 52b, 52c, 52d such that the second, third, and fourth actuators 82b, 82c, 82d are actuatable by one or more of the free fingers of the hand “H” of the clinician, depending, for example, on which hand “H” of the clinician is engaged with the handle controller 60.

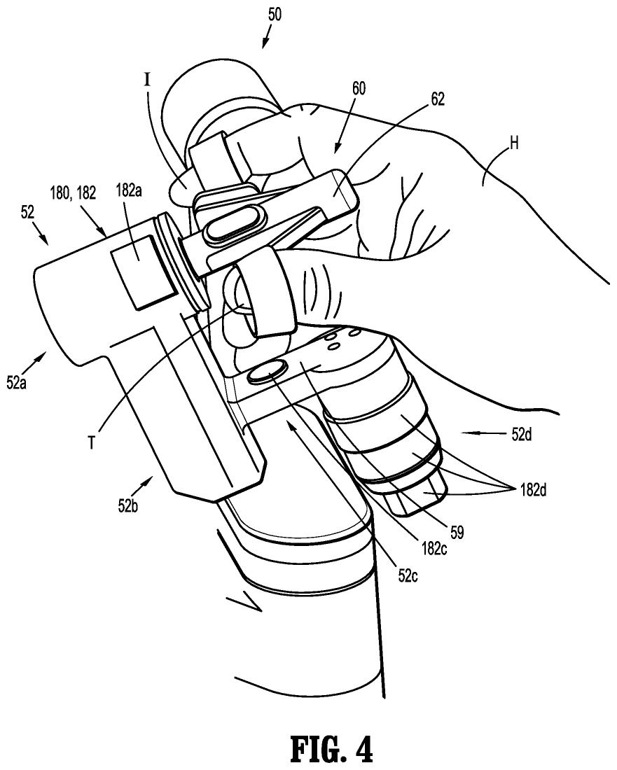

FIG. 4 illustrates another exemplary configuration of a gimbal actuation assembly 180. A first segment 52a of the first link 52 of the gimbal 50 supports a first actuator 182a that is disposed distal to the hand “H” of the clinician, the third segment 52c supports a third actuator 182c that is disposed on a top surface 59 of the third segment 52c which is located under the handle body 62 of the handle controller 60, and the fourth segment 52d supports a fourth actuator 182d. The first actuator 182a is shown as a touchpad, the third actuator 182c is shown as a multi-function control knob, and the fourth actuator 182d is shown as a multi-function rotation knob. The touchpad enables a clinician to swipe or peck at the first actuator 182 with the thumb “T” or index finger “I”, the multi-function control knob enables a clinician to effect various functions by, for example, rotating, pivoting, and/or pressing the control knob with one of the free fingers positioned adjacent to the top surface 59 of the third segment, and the multi-function rotation knob enables a clinician to effect various function by, for example, rotating one or more collars of rotation knob.

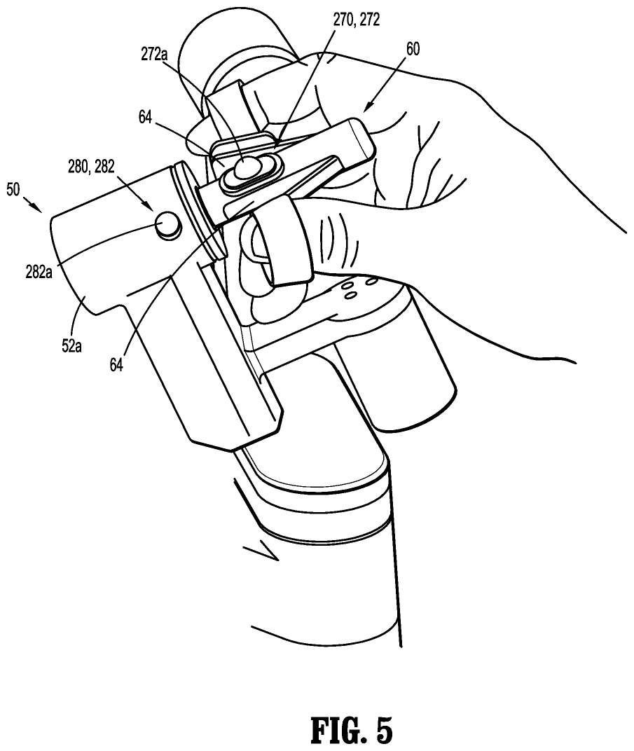

FIG. 5 illustrates yet another exemplary configuration of handle-mounted and gimbal-mounted actuator assemblies 270, 280. The handle controller 60 includes a first actuator 272a supported on the handle body 62 between the pinchers 64, and the gimbal 50 includes a first actuator 282a disposed on the first segment 52a of the first link 52. In such aspects, the handle-mounted and gimbal-mounted actuator assemblies 270, 280 are actuatable using the thumb “T” and/or index finger “I” of the clinician. The first actuator 272a of the handle-mounted actuator assembly 270 is shown as a trackball, and the first actuator 282a of the gimbal actuator assembly 280 is a joystick.

Although shown as including specific actuators in various specific locations about the handle controller and the first link of the gimbal, it should be understood various types and/or number of actuators may be placed in various locations about the handle controller and the gimbal depending upon the functional control desired or required for the robot system. Accordingly, it is envisioned that input devices of this disclosure may be configured to including handle-mounted and/or gimbal-mounted actuator assemblies in a variety of configurations that are suitable for use with a variety of different types of handle controllers and gimbals as is within the purview of those skilled in the art.

Additionally, some of all of the components of the gimbal and/or the handle controller of this disclosure may be modular so that different gimbals, handle controllers, or components of the gimbal and/or the handle controller may be utilized with the input devices of the robotic surgical system. For example, a connector between the gimbal and the control arm may allow a clinician to change the gimbal of an input device, a connector between the gimbal and the handle controller may allow a clinician to change the handle controller of the input device, and/or one or more connectors within the gimbal and/or the handle controller may allow for some of the components of the gimbal (e.g., the first, second, or third link) and/or the handle controller (e.g., the handle body or the grip components (e.g., the pinchers)) to be changed. The modularity of the gimbal and/or the handle controller allows a clinician to use a unique or custom input device for different surgical applications. The gimbal, the handle controller, or components thereof may be changed depending upon, for example, the desired size of the handle controller (e.g., for ergonomic fit with the hand of the clinician), the desired placement of the actuators (e.g., for ergonomic movement of the hand of the clinician), or the type of gimbal, handle controller, and/or actuator(s) desired for use to control a surgical instrument of the robotic surgical system (e.g., an input device configured to control and effect functions of a stapling surgical instrument, a vessel sealing surgical instrument, a grasping surgical instrument, etc.).

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Additionally, the elements and features shown and described in connection with certain aspects of the disclosure may be combined with the elements and features of certain other aspects without departing from the scope of the present disclosure, and that such modifications and variation are also included within the scope of the present disclosure. Therefore, the above description should not be construed as limiting, but merely as exemplifications of aspects of the disclosure and the subject matter of the present disclosure is not limited by what has been particularly shown and described. Thus, the scope of the disclosure should be determined by the appended claims and their legal equivalents, rather than by the examples given.

What is claimed is:

1. A surgical robot system comprising:
a robot system including a robotic arm having a surgical instrument coupled thereto;
a surgical console including an input device, the input device including:
a gimbal including first, second, and third links;
a handle controller coupled to the first link of the gimbal; and
a gimbal-mounted actuator assembly including a first actuator supported on the gimbal, the first actuator configured to effect a function of the surgical instrument coupled to the robotic arm; and
a control unit configured to control the robotic arm based on input commands from the input device.

2. The surgical robot system according to claim 1, wherein the first actuator of the gimbal-mounted actuator assembly is supported on the first link of the gimbal.

3. The surgical robot system according to claim 1, wherein the first link of the gimbal includes a first segment extending distally from the handle controller and a second segment extending downwardly from the first segment, the first actuator of the gimbal-mounted actuator assembly disposed on the first segment.

4. The surgical robot system according to claim 3, wherein the gimbal-mounted actuator assembly includes a second actuator supported on the second segment of the first link.

5. The surgical robot system according to claim 3, wherein the first link of the gimbal includes a third segment extending proximally from the second segment and under the handle controller.

6. The surgical robot system according to claim 5, wherein the gimbal-mounted actuator assembly includes a third actuator supported on the third segment.

7. The surgical robot system according to claim 5, wherein the first link of the gimbal includes a fourth segment extending downwardly from the third segment, and the gimbal-mounted actuator assembly includes a fourth actuator supported on the fourth segment.

8. The surgical robot system according to claim 1, wherein the handle controller includes a shaft and pinchers disposed on opposed sides of the shaft.

9. The surgical robot system according to claim 8, wherein the input device of the surgical console further includes a handle-mounted actuator assembly including a first actuator supported on the shaft of the handle controller.

10. The surgical robot system according to claim 1, wherein the first actuator of the gimbal-mounted actuator assembly is selected from the group consisting of push buttons, touch pads, rotation knobs, toggle switches, slide switches, rocker switches, rotary switches, trackpads, trackballs, and joysticks.

11. A surgical console for a surgical robot system, the surgical console comprising:
a control arm assembly including a plurality of input devices, at least one of the plurality of input devices including:
a gimbal including first, second, and third links;
a handle controller coupled to the first link of the gimbal; and
a gimbal-mounted actuator assembly including a first actuator supported on the gimbal.

12. The surgical console according to claim 11, wherein the first actuator of the gimbal-mounted actuator assembly is supported on the first link of the gimbal.

13. The surgical console according to claim 11, wherein the first link of the gimbal includes a first segment extending distally from the handle controller and a second segment extending downwardly from the first segment, the first actuator of the gimbal-mounted actuator assembly disposed on the first segment.

14. The surgical console according to claim 11, wherein the handle controller of the input device includes a shaft and pinchers disposed on opposed sides of the shaft.

15. The surgical console according to claim 14, wherein the input device further includes a handle-mounted actuator assembly including a first actuator supported on the shaft of the handle controller.

16. An input device for a surgical robot system, the input device comprising:
a gimbal including first, second, and third links;
a handle controller coupled to the first link of the gimbal; and
a gimbal-mounted actuator assembly including a first actuator supported on the gimbal.

17. The input device according to claim 16, wherein the first actuator of the gimbal-mounted actuator assembly is supported on the first link of the gimbal.

18. The input device according to claim 16, wherein the first link of the gimbal includes a first segment extending distally from the handle controller and a second segment extending downwardly from the first segment, the first actuator of the gimbal-mounted actuator assembly disposed on the first segment.

19. The input device according to claim 16, wherein the handle controller includes a shaft and pinchers disposed on opposed sides of the shaft.

20. The input device according to claim 19, further including a handle-mounted actuator assembly including a first actuator supported on the shaft of the handle controller.
